(19) United States
(12) Patent Application Publication
(10) Pub. No.: US 2018/0064404 A1
(43) Pub. Date: Mar. 8, 2018

(54) System and Method for Correcting Sleep Aberrations

(71) Applicants: Alex Zheng, Fremont, CA (US); Phil Ryder, Gates Mills, OH (US)

(72) Inventors: Alex Zheng, Fremont, CA (US); Phil Ryder, Gates Mills, OH (US)

(21) Appl. No.: 15/259,551

(22) Filed: Sep. 8, 2016

(51) Int. Cl. A61B 5/00; A61B 7/00; A61B 5/11; A61B 5/1455

(52) U.S. Cl. CPC A61B 5/746; A61B 7/003; A61B 5/4818; A61B 5/7278; A61B 5/11; A61B 5/7455; A61B 2562/0219; A61B 5/0022; A61B 5/7475; A61B 5/14551; A61B 5/6804; A61B 2562/0204; A61B 5/742

(57) ABSTRACT

In some embodiments, a method and system may detect snoring sounds of a sleeping patient through a microphone on the electronic device; analyze the snoring sounds to calculate a snore frequency and a snore volume of the sleeping patient, in response to the snore frequency exceeding a threshold frequency or the snore volume exceeding a threshold volume, sense an orientation of the electronic device, wherein the orientation is one of at least four different orientations; store the orientation as a snore-orientation; and by the one or more vibration units on the electronic device, vibrate if: (1) a current orientation of the electronic device matches the snore-orientation, and (2) the snore frequency exceeds the threshold frequency, or the snore volume exceeds the threshold volume.

### PRIORITY

This application claims the benefit of provisional patent application 62/216,515 filed on 10 Sep. 2015 and incorporated herein by reference.

### BACKGROUND

Sleep apnea is a type of sleep disorder characterized by pauses in breathing or instances of reduced breathing during sleep. Each pause in breathing, called an apnea, can last from at least ten seconds to several minutes, and may occur 5 to 30 times or more an hour. Similarly, each reduced breathing event, when accompanied by a corresponding reduction in blood oxygen saturation (minimum of 3 or 4% depending on the scoring guidelines), is called a hypopnea.

A related and more common issue among sleepers is snoring. Snoring is the vibration of respiratory structures and the resulting sound due to obstructed air movement during sleep. Snoring may cause sleep deprivation to both the snorer and those sharing a bed or room with the snorer. It is estimated that 30% of adults snore, and up to 60% of middle-aged men snore. Snoring most often occurs when a sleeper lays on her back because the airway is narrowed by the force of gravity causing her tongue to fall to the back of the throat during sleep. However, sleeping on one's side may reduce snoring because the base of the tongue will not collapse into the back of the throat, obstructing breathing. Therefore, sleeping on one's side is one way to prevent snoring.

### SUMMARY

In various embodiments, a snore prevention device may be worn by the sleeper during sleep. The snore prevention device may be attached directly to the sleeper's skin, or may be attached to the sleeper's clothing. The snore prevention device may include several sensors, including an accelerometer, a gyroscope, a microphone, a light sensor, among other sensors. The snore prevention device may monitor the sleeper's orientation while she sleeps, and may also monitor the sleeper's snore levels in terms of frequency and volume. The snore prevention device may detect the instances when the sleeper snores and also detect the sleeper's orientation while she snores (e.g., is the sleeper on her back, right-side, left-side, or stomach?). The snore prevention device may also include a vibration unit. The snore prevention device may be programmed to vibrate when it detects, via the snore prevention device's microphone, that the sleeper is snoring. The vibration may awake the sleeper briefly and act as a cue to change her sleeping position. For example, if the sleeper is laying on her back and is also snoring, the snore prevention device may vibrate. The vibration may cause the sleeper to wake up and shift her sleeping position (e.g., lay on her side).

The snore prevention device may also be programmed to monitor a person's sleep through the various sensors on the snore prevention device. The more still and quiet a sleeper is, the better she is sleeping. The snore prevention device may use this data, along with the orientation data gathered from the gyroscope and accelerometer, to learn a sleeper's best sleeping position. The snore prevention device may then vibrate to prompt the sleeper to assume her best sleeping position. For example, if a sleeper lies on her back but is restless as she sleeps (e.g., by snoring, fidgeting, or otherwise moving), the snore prevention device may detect that (1) the sleeper is on her back and (2) she is not sleeping well. The snore prevention device may vibrate to prompt the sleeper to sleep in a different position (e.g., on her left side). The snore prevention device may then monitor the sleeper and detect that the sleeper is sleeping much more soundly (e.g., she is not snoring or fidgeting). After repeated use, the snore prevention device may learn that the left side is the best position for this particular sleeper. Thus, when the sleeper falls asleep in any position beside her left side (e.g., on her back), the snore prevention device may vibrate to prompt the sleeper to lay on her left side.

### BRIEF DESCRIPTION OF THE DRAWINGS

FIG. 1 illustrates an example diagram showing an example system for performing an example method of prompting a sleeper to change sleeping position.

FIG. 2 illustrates an example block diagram showing the basic components of an example snore prevention device.

FIG. 3 illustrates an example user interface of an application associated with a snore prevention device.

FIG. 4 illustrates an example user interface of an application associated with a snore prevention device.

FIG. 5 illustrates an example user interface of an application associated with a snore prevention device.

### DETAILED DESCRIPTION

As will be appreciated by one of skill in the art, the example embodiments may be actualized as, or may generally utilize, a method, system, computer program product, or a combination of the foregoing. Accordingly, any of the embodiments may take the form of an entirely hardware embodiment, an entirely software embodiment (including firmware, resident software, microcode, etc.) for execution on hardware, or an embodiment combining software and hardware aspects that may generally be referred to as a “system.” Generally, the “system” will comprise a remotely located server with storage capability such as one or more databases that interact with a plurality of remote devices via a communication network such as the Internet, an intranet, or another communication network such as a cellular network. The smart devices that will be used in the location of the patient include any of a plurality of computing devices, such as smart phones, phablets, tablets, or personal computers, for example. The remote devices will execute software gone or more “apps”) that has been downloaded from the server to each of the remote devices to perform the functions described herein.

Furthermore, some of the embodiments may take the form of a computer program product on a computer-usable storage medium having computer-usable program code embodied in the medium configured for installation on a computing device for execution.

Today's smart devices (e.g., smart phones, watches, PDAs, tablets, phablets, personal computers (PCs), and even smart wearable devices) are widely available in the US and are very multi-functional, being capable of specialized programming for novel uses and applications. Most of these devices, in particular mobile devices such as smart phones, tablets, phablets or PDAs have a number of built-in sensors including: one or more microphones, a light sensor, accelerometer, and a gyroscope. Such sensor can also be used as inputs to a PC. These sensors can be utilized, along with specialized software, to create a new specialized device and method for diagnosing sleep apnea in a convenient and non-intrusive manner.

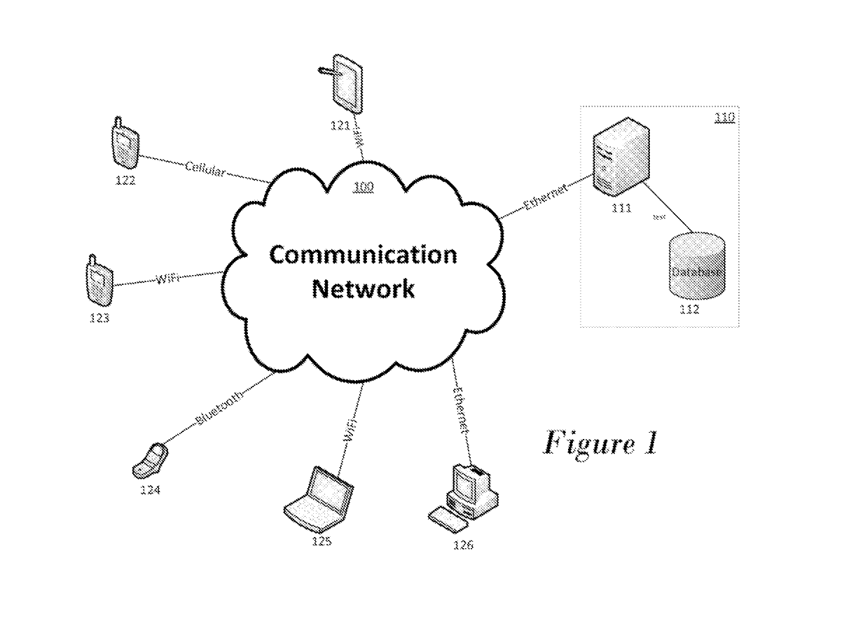

FIG. 1 shows an example system for implemented the disclosed diagnostic process. Various smart computing devices are used for individual patients to collect the sleep data for diagnosing sleep apnea. These smart devices may be replaced by a single-purpose dedicated snore prevention device. The smart devices may utilize any computing device capable of executing specialized software for collecting the sleep data, with such devices including such diverse devices as smartphones 122, 123, cell phones 124, tablets 121, laptops 125, PCs 126, and stand-alone electronic devices, among others, and which can connect to a communication network 100 such as the Internet using various communications protocols such as WiFi, cellular networks, Bluetooth, Ethernet, etc. These smart devices (or snore prevention device) may then communicate with one or more locally or remotely located analysis devices 110 including an analysis computer 110 and optionally a database 112 for receiving, storing, and performing an analysis on the data received and collected from the smart devices or snore prevention device. In particular, smart devices comprised of personal mobile computing devices (e.g., smart phones, tablets, PDAs, etc.) are particularly useful, considering their ubiquitous nature and configurability with customized applications. In some embodiments a snore prevention device communicates with an application on a mobile computing device (e.g., smartphone). The snore prevention device may record data and transmit the data to the mobile computing device, where it is either analyzed locally, or sent to analysis computer 110 or database 112 for analysis. Alternatively, the snore prevention device may be operable to analyze the data it gathers and then send its calculations to the mobile computing device for output on a user interface application.

Optionally, the functions of the snore prevention device, smart computing device, and analysis computer can be combined into either the snore prevention device or the smart computing device, avoiding the need of an intervening communication network. For example, a smart computing device with sufficient computing capability and storage (e.g., a smartphone may be used as a self-contained device to perform all parts of the data collection and diagnostic functions.

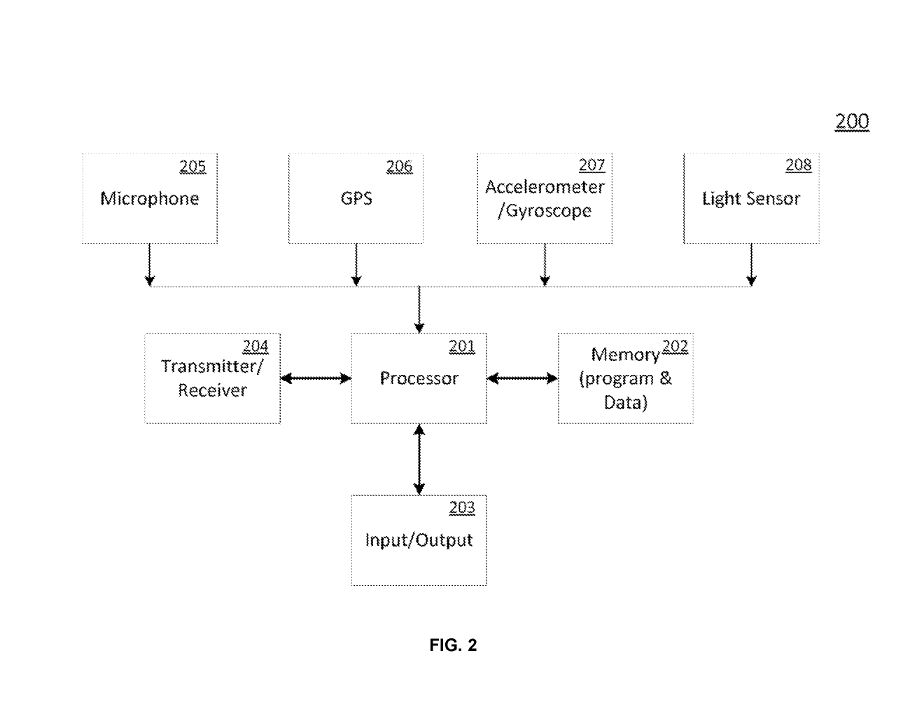

FIG. 2 illustrates an example block diagram showing the basic components of an example snore prevention device 200. The device may comprise one or more processors 201 and one or more memories 202 for storing embedded logic, software instructions, and data. The snore prevention device 200 may have a transmitter/receiver 204 for connecting to the communication network to transmit data to the analysis device 110 (FIG. 1) for performing the medical analysis the collected data. Alternatively, the transmitter/receiver may operate to transmit data to a mobile device associated with a patient. The data may be analyzed and organized and then displayed to show the patient various information, such as sleeping position, the amount of time the patient snored in the past night or week or longer, the number of “sleep events” that occurred in the past night or week or longer, and any other suitable information. A sleep event may be understood to mean a period of time When the patient was not breathing (e.g., an apnea).

The snore prevention device 200 may also includes one or more input/output interface 203 for communicating with a user, such as a touch screen, keypad, etc. The snore prevention device 200 may also have a plurality of input sensors, such as a microphone 205, GPS subsystem 206, accelerometer and gyroscope 207, and light sensor 208 with which it can collect data from its surroundings. Snore prevention device 200 may also include a vibration unit, which may vibrate when either snore prevention device 200 or a mobile computing device with a dedicated software application detects that the patient is snoring and in a snore-prone position (e.g., in a position where she is likely to snore, such as on her back).

In various embodiments, snore prevention device 200 may also include a pulse oximetry (“pulseOx”) sensor. The pulseOx sensor may comprise internal or external LEDs and a photocell. In some embodiments, if the LEDs are included in the snore prevention device 200 (internal LEDs), the pulseOx sensor may use a reflective approach to monitor a patient's oxygen saturation during sleep. In this case, snore prevention device 200 may be worn by the patient at a location where there is contact with the skin above a bone, and where there is a blood supply (e.g., forehead, sternum). If the LEDs are external, the pulseOx sensor may be worn on the finger. The signal and data gathered from the pulseOx sensor may be used in conjunction with the data gathered by snore prevention device 200 to corroborate a determination that an apnea event occurred, as well as to calculate or corroborate a “stopped breathing” time that may be calculated either by snore prevention device 200 or a mobile client device using a microphone.

In various embodiments, the microphone may be located on a mobile computing device associated with a patient, and snore prevention device 200 may include a gyroscope. With this arrangement, snore prevention device 200 may monitor the sleeper's orientation using the gyroscope and the mobile computing device (via a dedicated software application) may monitor the sleeper's snoring sounds. When the snoring sounds reach a threshold volume or a threshold frequency, the mobile computing device may access snore prevention device 200 and ask the sleeper's orientation (e.g., is the sleeper lying on her back, side, or stomach?). The mobile device may send a communication to snore prevention device 200 to vibrate when the snoring sounds reach a threshold volume or a threshold frequency. In some embodiments, snore prevention device 200 may monitor both snoring sounds and sleep orientation. If the snoring sounds reach a threshold frequency or a threshold volume, snore prevention device 200 may vibrate to prompt the sleeper to change sleeping positions e.g., move from her back onto her side).

In some embodiments, either snore prevention device 200 or a mobile computing device (via a dedicated software application) may learn which sleeping position is most likely to be associated with snoring, and vibrate to prompt the sleeper to change positions before she even starts snoring. As an example, if a sleeper, Anne, snores primarily when she is sleeping on her back, snore prevention device 200 or a mobile computing device may detect this pattern and may be programmed to automatically vibrate once the gyroscope detects that Anne is sleeping on her back. Snore prevention device 200 may be programmed to automatically vibrate once Anne has snored in a particular orientation (e.g., on her back) after a particular amount of times. For example, if Anne snores five times when she lays on her back, snore prevention device 200 may automatically vibrate the next time Anne sleeps on her back even if Anne has not started snoring yet.

In some embodiments, the user (e.g., sleeper or sleeping patient) may wear snore prevention device 200. This may be how snore prevention device 200 detects the user's sleeping orientation (e.g., whether the sleeper is sleeping on her back, right side, left side, or stomach). Snore prevention device 200 may be worn on the back of the neck, just under the cervical vertebrae (e.g., where the tag to a t-shirt usually goes). Snore prevention device 200 may adhere directly to the skin or may be attached to a shirt (e.g., by VELCRO or any other suitable connection) that the sleeper may wear during sleep.

Together, the snore prevention device 200 with the installed specialized software application installed in memory 202 that can be executed by the processor 201 to configure the device to act as a specialized machine for collecting data from the patient and the patient's surroundings during a monitoring period, in this case while the patient is sleeping.

Any suitable computer usable (computer readable) medium may be utilized for storing the different specialized software applications that are executed by the analysis device 110 and the snore prevention device 200, respectively. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), cloud storage (remote storage, perhaps as a service), or other tangible optical or magnetic storage device; or transmission media such as those supporting the Internet or an intranet.

The specialized software applications are comprised of computer program code specifically configured for carrying out operations of the example embodiments (i.e., for the snore prevention devices and analysis device(s) of a given system) may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perk Smalltalk, C++, Object Pascal, C#, Swift, or the like, artificial intelligence languages such as Prolog, a real-time embedded language such as Ada, or even more direct or simplified programming using ladder logic, an Assembler language, or directly programming using an appropriate machine language. Web-based languages such as HTML or any of its many variants may be utilized. Graphical objects may be stored using any graphical storage or compression format, such as bitmap, vector, metafile, scene, animation, multimedia, hypertext and hypermedia, VRML, and other formats could be used. Audio storage could utilize any of many different types of audio and video files, such as WAV, AVE MPEG, MP3, MP4, WMA, FLAG, MOV, among others. Editing tools for any of these languages and/or formats can be used to create the software.

The computer program instructions of the software and/or scripts comprising the code may be provided to the respective computing device (e.g., a smartphone, tablet, phablet, PC or other device) which includes one or more programmable processors or controllers, or other programmable data processing apparatus, which executes the instructions via the processor of the computer or other programmable data processing apparatus for implementing the functions/acts specified in this document. It should also be noted that, in some alternative implementations, the functions may occur out of the order noted herein. The software applications can be downloaded to the respective devices in a conventional manner, and could be provided by a software vending site such as an Android or Apple app store, for example.

The specialized software application is installed in the smart computing device (in particular a personal computing device that is mobile and portable) for execution that causes the snore prevention device to collect and record sensor data from near or on a patient while the patient is sleeping. The specialized software is executed on the smart computing device to perform a diagnostic method that determines a sleep index that is highly correlated with AHI for diagnosing sleep apnea in the patient. This diagnostic method is based on a recording of the background noises near the patient, including the snoring and other night sounds generated by the patient. Additional data can also be collected, such as light or patient motion information, for example. The diagnostic method is adaptive in terms of the number of sensors available on the snore prevention device.

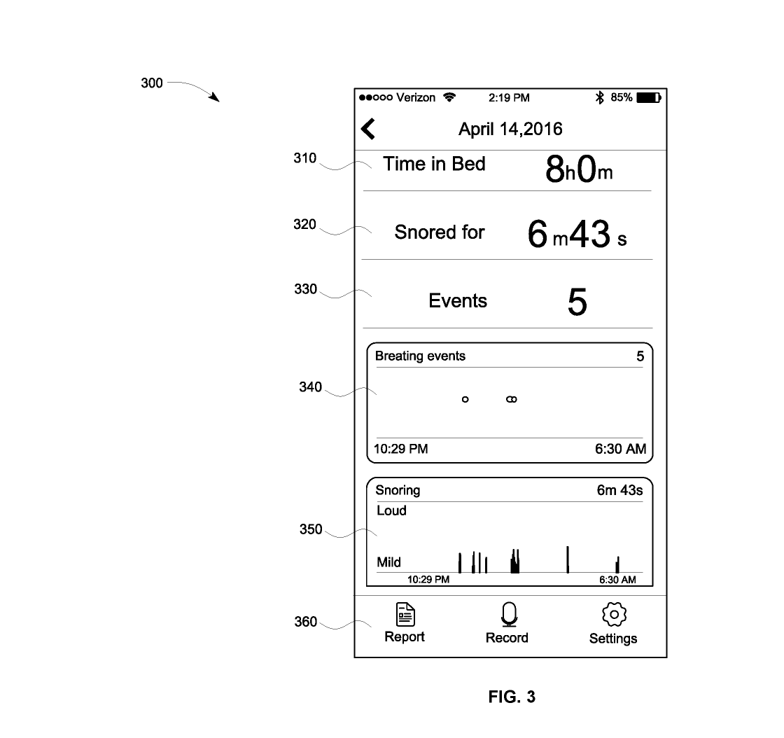

FIG. 3 illustrates an example user interface 300 of an application associated with a snore prevention device. User interface 300 may display a summary of a single night of sleep by a user. User interface 300 may include various panels of information, including but not limited to time in bed panel 310, snored for panel 320, sleeping events panel 330, breathing events graph 340, snore graph 350, and action panel 360. Time in bed panel 310 may display the amount of time the user spent in bed during a particular night, or the amount of time the user spent asleep during a particular night. Snore prevention device 200 may be able to determine when the user is asleep by monitoring the user's movement via the accelerometer included in snore prevention device 200. Snored for panel 320 may indicate how long the user snored for during the particular night, and may display this information as an amount of time (e.g., as hours and minutes), or as a percentage of the total time asleep or time in bed. As an example, if a user spent 8 hours in bed and snored for 6 of those hours, snored for panel 320 may simply display 6 hours, or may display 75%, since the user snored 75% of the time she was in bed. Sleeping events panel 330 may display the number of sleeping events that occurred during the particular night. Breathing events graph 340 may display, graphically, the time of night that the sleeping events occurred. Snore graph 350 may display the number of times the user snored during the night along with an indication of how loud the snoring was. Action panel 360 may allow the user to view different reports (e.g., snoring patterns for the previous month), or may allow the user to manually record audio, or may allow the user to adjust various settings in the application.

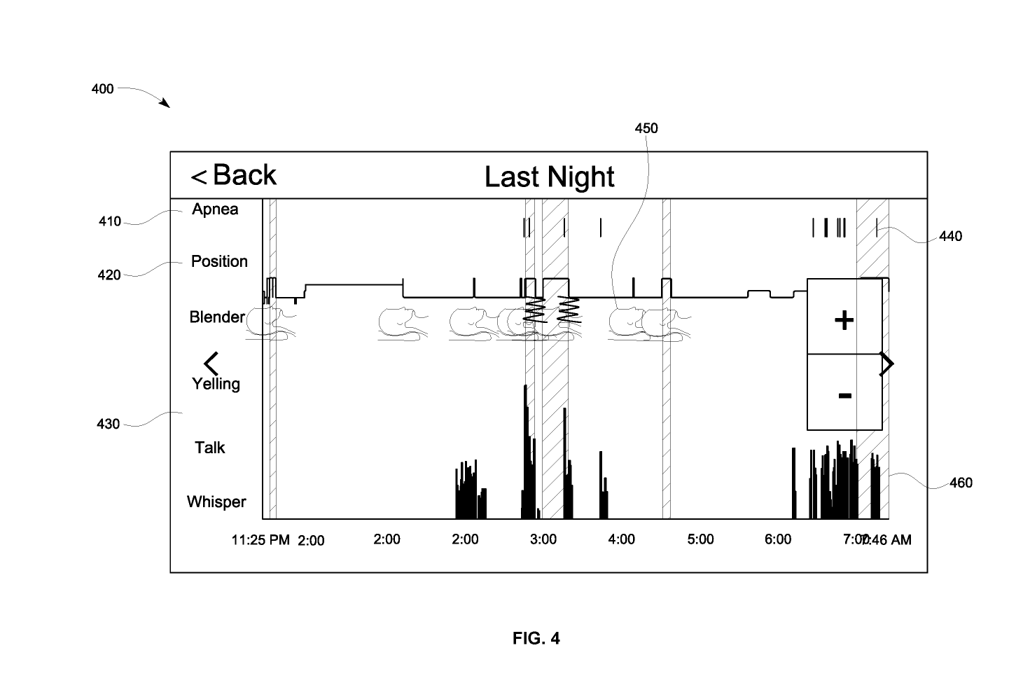

FIG. 4 illustrates an example user interface 400 of an application associated with a snore prevention device. The application may be the same application discussed in reference to FIG. 3, and may be another aspect of the application. User interface 400 may include an apnea event row 410, a position row 420, and one or more noise indicator rows 430. User interface 400 may also include one or more head position indicators 450. Apnea event row 410 may include one or more apnea event indicators 440. Apnea event indicators 440 may represent apnea events that occurred during the night while a patient was sleeping. Position row 420 may indicate the sleeping position of the patient (e.g., prone, supine, left side, right side) during the night. The x-axis of user interface 400 may indicate the time of night. For example, if the x-axis indicates that the time of night was 3:00 AM, and directly above 3:00 AM, one or more apnea event indicators 440 are displayed on apnea event row 410 and position row indicates (optionally via position indicators 450) that the patient was sleeping on her back, this may indicate that at 3:00 AM the patient was sleeping on her back and one or more apnea events occurred. This information may be helpful to the patient because it may indicate that an increased number of apnea events occur when she sleeps on her back. Additionally, noise indicator rows 430 may indicate how loud the environment was throughout the night. Noise may be the result of outside noise (e.g., a dog barking, an ambulance with its sirens on driving by), or may be the result of the patient snoring. As discussed herein, snore prevention device 200 or mobile computing device (via a dedicated software application) may be able to discern between the patient's snoring and other types of noise (e.g., dog barking, ambulance) by analyzing the sound waveforms recorded by a microphone. Snore prevention device 200 or mobile computing device may have various sound waveforms of snoring sounds stored on the device or may learn what a particular patient's snoring waveforms looks like through repeated use. Either way, snore prevention device 200 or mobile computing device may be able to compare the incoming sound waveforms to the snoring waveforms. If the two waveforms are substantially the same, snore prevention device 200 or mobile computing device may conclude that the incoming sounds are snoring sounds. If the two waveforms are different enough to pass a threshold difference, snore prevention device 200 or mobile computing device may conclude that the incoming sounds are something other than snoring sounds. This may help snore prevention device 200 or the mobile computing device in analyzing the patient's sleep index, and in triggering the vibration unit in snore prevention device 200 to prompt the patient to adjust her sleeping position.

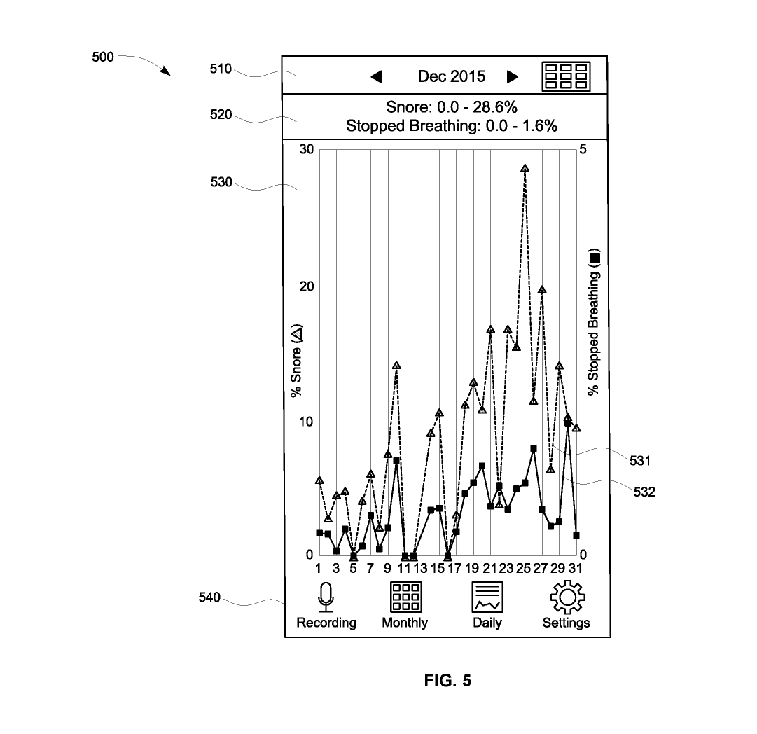

FIG. 5 illustrates an example user interface 500 of an application associated with a snore prevention device. The application may be the same application discussed in reference to FIGS. 3 and 4, and may be another aspect of the application. User interface 500 may display a periodical summary of the patient's sleeping pattern over a particular amount of time (e.g., the previous week, month, year). User interface 500 may include a date bar 510, an overview bar 520, a graph 530, and a selection bar 540. Date bar 510 may display the date of the relevant time period. Overview bar 520 may display particular key metrics either set by the user or that are predetermined. Example key metrics include the amount of time spent snoring, the percentage of sleep time the patient spent snoring, the number of apnea events that occurred during the relevant time period, the percent of time the patient slept restlessly or restfully, and any other suitable metric. Graph 530 may include one or more metrics, such as snore metric 531 and apnea metric 532. Snore metric 531 may indicate, graphically, the percentage of sleep time a patient snoring during the relevant time period. This may be helpful because it may indicate if the patient spends more time snoring at particular times during the month. Apnea metric 532 may indicate, graphically, the number of sleep apnea events that occurred per night during the relevant time period. This may be helpful because it may indicate if the patient experiences more sleep apnea events at particular times during the month. The patient may be able to use this information to make more informed decisions about sleep and lifestyle. For example, if the patient is particularly stressed out at work during a given week, the patient may check graph 530 to determine whether snoring and sleep apnea events have risen during the given week. If they have, she may conclude that there is a correlation between stress at work and snoring and sleep apnea. Selection bar 540 may enable the user to perform various operations inside the application, such as record manually, see monthly or daily metrics, and adjust various settings associated with the application.

The smart computing device (e.g., smartphone) executes the specialized software application to guide the user (patient) through the following steps to ensure that the recording is of the highest quality possible: (1) Calibrating: (a) the light sensors, (b) movement (e.g., accelerometer) sensors, and (c) and microphone sensors (any other sensors to be used may be similarly calibrated as well).; (2) The user is reminded by the device, such as by a textual or audio message, for example, that only the user (patient) should be sleeping in the room at night during the process or only the user snores when there are multiple people sleeping in the room; (3) The recording/diagnostic application is started.

The user can choose to place the smart computing device or a portion thereof on himself/herself during sleep (when such a device or the portion thereof is properly sized for such use), or anywhere nearby (such as when the device is a larger and/or heavier computing device). In situations where the device (or at least a portion having a sensor for detecting motion/movement) is mounted, worn, or otherwise placed on the user, the device may be better able to determine any movement of the user (patient) during the night, which could prove useful in determining the periods where the sound recording may be most accurate for use in the diagnosis.

During the night, an application that is part of the specialized software executes on the snore prevention device to record the night sounds while the patient is sleeping, along with recording any other data that is detected during this process. The data is correlated with the time of the recording.

Upon the completion of the data recording process, such as by the end of the night, or after a certain number of hours of recording (which may be predetermined or user selectable), the snore prevention device transmits the recorded data to another analysis device executing additional specialized to act as an analysis engine for storage and analysis of the recorded data. The analysis engine may execute an algorithm to determine the sleep index using various rules. Optionally, signal processing may be performed on the detected and/or recorded audio data to prevent the reconstruction of the original sounds (e.g., voices) to protect patient privacy but at the same time maintain the desired information to diagnose the medical condition (e.g., sufficient for snore and apnea detection).

Specifically, the analysis to determine the sleep index (i.e., determine whether sleep apnea is occurring and the snoring patterns of the patient) involves: (1) Determining the wake period where the data indicates that there is speaking or other noises/sounds of certain minimum duration indicating that the patient is likely awake; (2) Determining an apnea event where the data indicates that a limited duration of no snore is followed by one or a few snores, which are then followed by another limited period of no snore; (3) Determining an apnea event where the data indicates that there is a crescendo of limited duration in snore, followed or preceded by no snore event of a limited duration; (4) Determining an apnea event where the data shows that there is no snore of limited duration; (5) Determining an apnea event where the data shows that there is a diminuendo of limited duration in snore, followed or preceded by no snore of a limited duration; and (6) Determining an apnea event where the data shows that there is a crescendo or diminuendo in the snore of limited duration.

For each pattern described above, the analysis may compute the probability of an apnea event by taking into account the following additional information: (1) The determined snore pattern and characteristics throughout the sleep; (2) a current determined snore amplitude and duration, relative to nearby periods before and after the current snore pattern; and (3) Other sensor recordings—for example, if there is recording for the light sensor and the light is determined to be ON during the period when there is no snore in contrast to determined to be OFF at other times, then the probability of an apnea event is near zero.

After all the patterns are determined and their probabilities computed, an overall sleep index and its confidence interval may be computed based on the determined apnea events, which is used to diagnose sleep apnea, if present.

If a recording for data from the gyroscope sensor is available, this data can be used to determine the sleep index for each sleep position as the sensor detects that the person has moved or otherwise changed position. In this way, desirable vs. undesirable sleep positions can also be determined.

As an extension, the snore prevention device can be configured using the specialized software to detect many different (e.g., all) sounds during the process. This can be used to compute a sound level distribution and determine the base background noise. Such a process can be carried out over multiple intervals so that a base background noise level is determined for each interval (for example, the background noise at 1 am differs from 7 am). Then, the base background noise can be subtracted or otherwise extracted from the desired sound data. The desired data sound level can then be scaled based on the calibrated sound level so that the desired sound level is more independent of the specific device and specific patient. Furthermore, the intervals where the sound levels are above some threshold can be determined, which can be used as the sound intervals for determining the snore periods and ultimately for detecting the apneas.

Another extension may be to detect and categorize snores and other sounds. The detected sounds can be sorted into three categories based on their characteristics, with example categories being: (1) Snores, determined when their durations are within a certain range (e.g., between 0.1 and 2 seconds) and when they occur at a frequency between ⅙ (one snore per six seconds) and ⅓ (one snore per three seconds) when apnea events are excluded. Also snores may appear in a large number. (2) Talk: gaps between sounds tend to be small; and (3) Sudden sounds (e.g., bed frame squeezing sound from body movement). Usually there is no sound before and after sudden sounds. Various heuristics may also be deployed to help categorize the sounds (e.g., snores do not follow talk immediately.

Many other example embodiments can be provided through various combinations of the above described features. Although the embodiments described above use specific examples and alternatives, it will be understood by those skilled in the art that various additional alternatives may be used and equivalents may be substituted for elements and/or steps described herein, without necessarily deviating from the intended scope of the application. Modifications may be necessary to adapt the embodiments to a particular situation or to particular needs without departing from the intended scope of the application. It is intended that the application not be limited to the particular example implementations and example embodiments described herein, but that the claims be given their broadest reasonable interpretation to cover all novel and non-obvious embodiments, literal or equivalent, disclosed or not, covered thereby. In this disclosure, it may be understood that sleeper, user, patient, and sleeping patient all refer to the user of snore prevention device 200.

What is claimed is:

1. A method, comprising:
by the electronic device, detecting snoring sounds of a sleeping patient through a microphone on the electronic device;
by the electronic device, analyzing the snoring sounds to calculate a snore frequency and a snore volume of the sleeping patient;
by the electronic device, in response to the snore frequency exceeding a threshold frequency or the snore volume exceeding a threshold volume, sensing an orientation of the electronic device, wherein the orientation is one of at least four different orientations;
by the electronic device, storing the orientation as a snore-orientation; and
by a vibration unit on the electronic device, vibrating if:
a current orientation of the electronic device matches the snore-orientation, and
the snore frequency exceeds the threshold frequency, or the snore volume exceeds the threshold volume.

2. The method of claim 1, further comprising sending the snore-frequency, snore volume, and snore orientation to a user interface associated with the electronic device.

3. The method of claim 1, wherein the snore frequency comprises the number of snoring sounds the sleeping patient makes per minute.

4. The electronic device of claim 1, wherein the snore volume, the snore frequency, and the orientation is displayed on a user interface of a client device associated with the sleeping patient.

5. The method of claim 1, wherein:
a first one of the four orientations correspond to the sleeping patient sleeping in a prone position;
a second one of the four orientations correspond to the sleeping patient sleeping in a supine position;
a third one of the four orientations correspond to the sleeping patient sleeping in a left-side sleeping position; and
a fourth one of the four orientations correspond to the sleeping patient sleeping in a right-side sleeping position.

6. The method of claim 1, further comprising detecting one or more sleep events, the sleep events comprising a period of sleep apnea above a threshold period.

7. The method of claim 1, wherein the threshold frequency or snore frequency is determined by the sleeping patient prior to the sleeping patient falling asleep.

8. An electronic device, comprising:
one or more processors;
one or more sensors, the sensors comprising an accelerometer, a gyroscope, and a microphone; and
one or more vibration units:
logic encoded in one or more computer-readable tangible storage media that, when executed by the one or more processors, is operable to:
detect snoring sounds of a sleeping patient through a microphone on the electronic device;
analyze the snoring sounds to calculate a snore frequency and a snore volume of the sleeping patient;
in response to the snore frequency exceeding a threshold frequency or the snore volume exceeding a threshold volume, sense an orientation of the electronic device, wherein the orientation is one of at least four different orientations;
store the orientation as a snore-orientation; and
by the one or more vibration units on the electronic device, vibrate if:
a current orientation of the electronic device matches the snore-orientation, and
the snore frequency exceeds the threshold frequency, or the snore volume exceeds the threshold volume.

9. The electronic device of claim 8, wherein the logic is further operable to send the snore-frequency, snore volume, and snore orientation to a user interface associated with the electronic device.

10. The electronic device of claim 8, wherein the snore frequency comprises the number of snoring sounds the sleeping patient makes per minute.

11. The electronic device of claim 8, wherein the snore volume, the snore frequency, and the orientation is displayed on a user interface of a client device associated with the sleeping patient.

12. The electronic device of claim 8, wherein:
a first one of the four orientations correspond to the sleeping patient sleeping in a prone position;
a second one of the four orientations correspond to the sleeping patient sleeping in a supine position;
a third one of the four orientations correspond to the sleeping patient sleeping in a left-side sleeping position; and
a fourth one of the four orientations correspond to the sleeping patient sleeping in a right-side sleeping position.

13. The electronic device of claim 8, wherein the logic is further operable to detect one or more sleep events, the sleep events comprising a period of sleep apnea above a threshold period.

14. The electronic device of claim 8, wherein the threshold frequency or snore frequency is determined by the sleeping patient prior to the sleeping patient falling asleep.

15. A system, comprising:
one or more processors;
one or more sensors, the sensors comprising an accelerometer, a gyroscope, and a microphone; and
one or more vibration units;
logic encoded in one or more computer-readable tangible storage media that, when executed by the one or more processors, is operable to:
detect snoring sounds of a sleeping patient through a microphone on the electronic device;
analyze the snoring sounds to calculate a snore frequency and a snore volume of the sleeping patient;
in response to the snore frequency exceeding a threshold frequency or the snore volume exceeding a threshold volume, sense an orientation of the electronic device, wherein the orientation is one of at least four different orientations;
store the orientation as a snore-orientation; and
by the one or more vibration units on the electronic device, vibrate if:
a current orientation of the electronic device matches the snore-orientation, and
the snore frequency exceeds the threshold frequency, or the snore volume exceeds the threshold volume.

16. The system of claim 15, wherein the logic is further operable to send the snore-frequency, snore volume, and snore orientation to a user interface associated with the electronic device.

17. The system of claim 15, wherein the snore frequency comprises the number of snoring sounds the sleeping patient makes per minute.

18. The system of claim 15, wherein the snore volume, the snore frequency, and the orientation is displayed on a user interface of a client device associated with the sleeping patient.

19. The system of claim 15, wherein:
a first one of the four orientations correspond to the sleeping patient sleeping in a prone position;
a second one of the four orientations correspond to the sleeping patient sleeping in a supine position;
a third one of the four orientations correspond to the sleeping patient sleeping in a left-side sleeping position; and
a fourth one of the four orientations correspond to the sleeping patient sleeping in a right-side sleeping position.

20. The system of claim 15, wherein the logic is further operable to detect one or more sleep events, the sleep events comprising a period of sleep apnea above a threshold period.
